(19) United States
(12) Patent Application Publication
(10) Pub. No.: US 2015/0342553 A1
(43) Pub. Date: Dec. 3, 2015

(54) IMAGING APPARATUS

(71) Applicant: CANON KABUSHIKI KAISHA, Tokyo (JP)

(72) Inventors: Takashi Sato, Tokyo (JP); Youjirou Hiratsuka, Yokohama-shi (JP)

(21) Appl. No.: 14/719,465

(22) Filed: May 22, 2015

(30) Foreign Application Priority Data
May 27, 2014 (JP) 2014-109428

(51) Int. Cl. A61B 6/00; G01T 1/161

(52) U.S. Cl. CPC A61B 6/5258; G01T 1/161

(57) ABSTRACT

An imaging apparatus comprises a housing including, on a side surface, at least one portion lower in magnetism shielding performance than a remaining portion of the housing, and configured to contain an image detector. The imaging apparatus includes a magnetic material that is arranged at a position between the image detector and the side surface including the portion, lower in magnetism shielding performance, of the housing, and a side of a rear surface of the image detector.

### BACKGROUND OF THE INVENTION

1. Field of the Invention

The present invention relates to an imaging apparatus.

2. Description of the Related Art

Conventionally, apparatuses that irradiate a target object with X-rays and detect the intensity distribution of the X-rays having passed through the target object to obtain the X-ray image of the target object are widely used in the fields of industrial non-destructive inspection and medical diagnosis. Such a digital X-ray imaging apparatus is an X-ray imaging apparatus using a semiconductor process technique. More specifically, small pixels each formed from a photoelectric converter, a switching element, and the like are two-dimensionally arrayed in the light receiving means of the digital X-ray imaging apparatus. The light receiving means detects, as an electrical signal, light converted from X-rays by a scintillator. The light receiving means of the digital X-ray imaging apparatus has a wider dynamic range in comparison with an imaging system using a silver halide film, and can obtain an X-ray captured image at a lower dose. The digital X-ray imaging apparatus has advantages in which chemical processing is unnecessary and output of a captured image can be instantaneously confirmed on a monitor or the like, unlike the imaging system using the silver halide film.

Since the X-ray detector of the digital X-ray imaging apparatus detects a weak analog signal, the following problem arises. In an imaging room in a hospital or the like, an apparatus that generates an X-ray, and another diagnosis inspection apparatus are arranged together with the digital X-ray imaging apparatus. In this environment, large-power devices, and a medical diagnosis device that handles a very weak signal coexist. It is becoming a problem recently that unwanted electromagnetic energy that is unnecessarily generated or leaks from these large-power devices causes a trouble regarding so-called electromagnetic interference (EMI), such as operation interference or malfunction of another device.

Examples of external noise that influences the digital X-ray imaging apparatus are radiation noise and conduction noise from another device. As for the conduction noise, a measure can be relatively easily taken by filter enhancement of the power supply system or the like. However, the radiation noise is electromagnetic field noise radiated into a space, and comes in from various directions in accordance with the installation/use state of the digital X-ray imaging apparatus, so it is difficult to take a measure. A large-power device, inverter X-ray generation apparatus, and the like generate magnetic field noise of 1 kHz to 100 kHz in a relatively low frequency band. A shield measure against AC magnetic field noise in such a frequency band is generally difficult.

When the AC magnetic field noise is superimposed on the X-ray detector of the digital X-ray imaging apparatus, horizontal-striped noise appears periodically in a captured image. This phenomenon is called line noise or line artifact noise. This is because, when sampling and holding a signal line, induction noise generated by an external AC magnetic field is superimposed on a signal, the phase relationship between the noise and the reading period sequentially shifts for every line, and the noise appears in a captured image as a beat of a frequency. Since the line noise is superimposed on a captured image, it may degrade the image quality and lead to misdiagnosis of a doctor in the case of a medical image, resulting in a serious problem.

Under these circumstances, necessity is growing for a structure in which internal electrical components and detection signals are hardly influenced by external electromagnetic noise in handling of a weak current in the digital X-ray imaging apparatus. Especially, the digital X-ray imaging apparatus increasingly needs to have a structure that is hardly influenced by AC magnetic field noise in a relatively low frequency band of 1 kHz to 100 kHz, which is AC magnetic field noise from a large-power device or the like.

Conventionally, for the housing of the digital X-ray imaging apparatus, there is proposed a shielding structure of six surfaces in which the digital X-ray imaging apparatus is completely surrounded by a conductive or magnetic exterior housing so no external magnetic field enters the inside of the housing. Japanese Patent Laid-Open No. 2004-177250 proposes a housing in which a scattered X-ray removal grid, a grid holding portion, and a housing are formed from conductive members to obtain conduction between all components and form an electrically enclosed structure. Japanese Patent Laid-Open No. 2005-249658 proposes a housing with an enclosed structure in which the whole exterior housing is surrounded by a high-permeability material.

However, in the structure that obtains conduction by a spring member or the like at the scattered X-ray removal grid portion that is inserted/removed, as disclosed in Japanese Patent Laid-Open No. 2004-177250, the contact resistance changes owing to aged deterioration of the spring or the like, it becomes difficult to obtain perfect conduction, and the connection reliability becomes poor. When no conduction is obtained owing to aging or the like, this state is equivalent to the presence of a gap or opening, an external magnetic field enters the inside of the housing and noise appears in a captured image.

In the arrangement disclosed in Japanese Patent Laid-Open No. 2005-249658, it is difficult to form an enclosed structure in terms of assembly in manufacturing and maintenance in the market. Since the high-permeability material of the enclosed structure requires a thickness of 1 to 3 mm or more with respect to an AC magnetic field in the relatively low frequency band of 1 kHz to 100 kHz, the cost of the overall product remarkably rises, and the weight also greatly increases.

### SUMMARY OF THE INVENTION

The present invention has been made to solve the above problem, and provides an imaging apparatus in which an image detector contained inside a housing is hardly influenced by external noise even in a structure in which a gap or opening is formed in the housing of the imaging apparatus.

According to one aspect of the present invention, there is provided an imaging apparatus comprising a housing including, on a side surface, at least one portion lower in magnetism shielding performance than a remaining portion of the housing, and configured to contain an image detector, wherein the imaging apparatus includes a magnetic material, and the magnetic material is arranged at a position between the image detector and the side surface including the portion, lower in magnetism shielding performance, of the housing, and a side of a rear surface of the image detector.

Further features of the present invention will become apparent from the following description of exemplary embodiments (with reference to the attached drawings).

### BRIEF DESCRIPTION OF THE DRAWINGS

FIG. 1 is a view showing the structure of an imaging apparatus according to the first embodiment;

FIGS. 2A to 2C are views for explaining the influence of external magnetic field noise according to the first embodiment;

FIGS. 3A and 3B are views for explaining the operation of a housing structure according to the first embodiment;

FIG. 4 is a view showing the structure of an imaging apparatus according to application example 1-1;

FIG. 5 is a graph for explaining the effect of application example 1-1;

FIG. 6 is a view showing the structure of an imaging apparatus according to application example 1-2;

FIG. 7 is a view showing the structure of an imaging apparatus according to application example 1-2;

FIG. 8 is a view showing the structure of an imaging apparatus according to application example 1-3;

FIG. 9 is a view showing the structure of an imaging apparatus according to application example 1-3;

FIG. 10 is a view showing the structure of an imaging apparatus according to application example 1-4;

FIG. 11 is a view showing the structure of an imaging apparatus according to application example 1-4;

FIG. 12 is a view showing the structure of an imaging apparatus according to the second embodiment;

FIGS. 13A to 13C are views for explaining the influence of external magnetic field noise according to the second embodiment;

FIG. 14 is a view for explaining the operation of the housing structure according to the second embodiment;

FIGS. 15A and 15B are views showing the structure of an imaging apparatus according to the third embodiment;

FIGS. 16A to 16C are views for explaining the influence of external magnetic field noise according to the third embodiment;

FIGS. 17A and 17B are views for explaining the operation of a housing structure according to the third embodiment;

FIG. 18 is a view showing the structure of an imaging apparatus according to application example 3-1;

FIG. 19 is a graph for explaining the effect of application example 3-1; and

FIG. 20 is a view showing the structure of an imaging apparatus according to application example 3-2.

### DESCRIPTION OF THE EMBODIMENTS

### First Embodiment

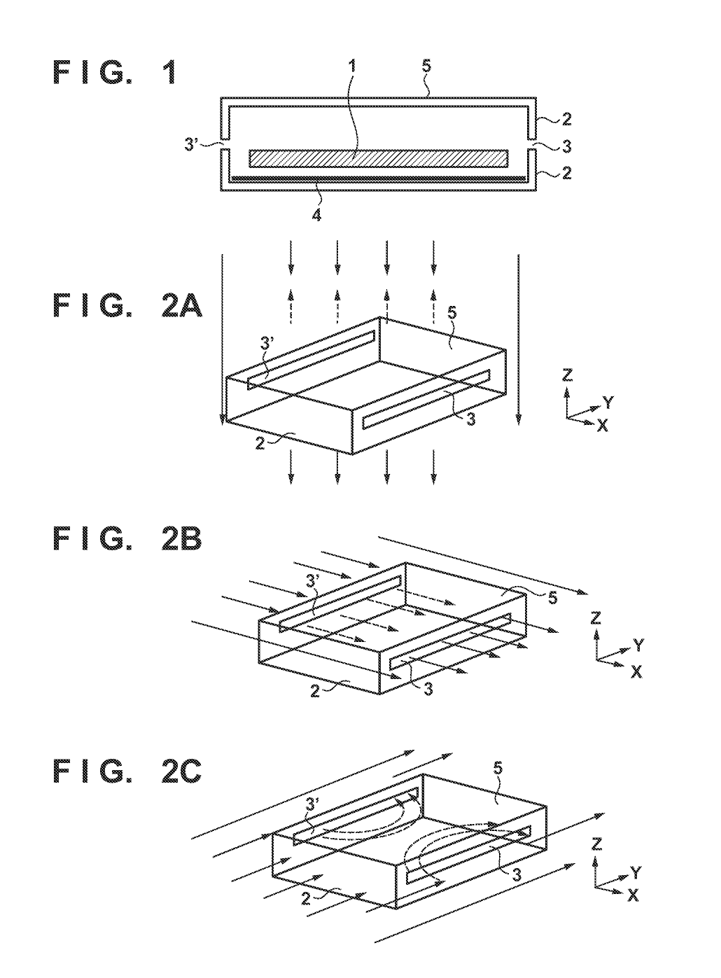

FIG. 1 shows the structure of an imaging apparatus according to the first embodiment. An image detector 1 is contained in a conductive housing 2 having an imaging surface 5 at a position facing the image detector 1. An opening 3 is formed in a side surface of the housing 2, and an opening 3′ is also formed in a side surface facing the opening 3. In this embodiment, an electrical or physical opening formed at the peripheral portion of the housing is an example of a portion lower in magnetism shielding performance than the remaining region. Hence, a structure other than the opening may also be formed as long as it is a portion lower in magnetism shielding performance than the remaining region. The image detector 1 is a device that obtains digital radiation image data, for example, it is an X-ray detector. A planar magnetic material 4 wider than the projection area of the image detector 1 is arranged on the rear surface of the image detector 1. In this embodiment, the housing 2 is made of a conductive metal generally used in the exterior housing of a product, such as aluminum, stainless steel, or a steel sheet. The magnetic material 4 is made using a permalloy, amorphous alloy, FINEMET®, ferrite, or the like, which is a magnetic material having a relative permeability of 1,000 to 200,000 in a frequency band of 1 kHz to 100 kHz.

Next, the vector components of magnetic fields externally entering the inside of the housing will be explained with reference to FIGS. 2A to 2C. FIGS. 2A to 2C are views for explaining the influence of external magnetic field noise according to this embodiment. In FIGS. 2A to 2C, the image detector 1 and magnetic material 4 contained inside the housing 2 in FIG. 1 are omitted, in order to explain a magnetic field entering the inside of the housing when the external magnetic field comes in.

Magnetic fields from various directions come into the imaging apparatus in accordance with the installation position and the use state under the influence of radiation of a magnetic field from a device installed nearby or a large-power device, or leakage of a magnetic field. A magnetic field actually coming into the inside of the housing is an AC component. In FIGS. 2A to 2C, to clarify the explanation, a magnetic field vector is expressed by arrows in one direction, and an external magnetic field is explained using spatial vectors along three, X-, Y-, and Z-axes. In the following description, a magnetic field of a vertical component perpendicularly coming into the imaging surface 5 is a Z component, and magnetic field components that are perpendicular to the Z component and perpendicularly come into the side surfaces of the housing 2 are X and Y components.

FIG. 2A shows a case in which a magnetic field of the Z component perpendicularly coming into the imaging surface 5 irradiates the housing 2, as indicated by arrows of solid lines. In FIG. 2A, when the magnetic field of the Z component perpendicularly coming into the imaging surface 5 irradiates the housing 2, as indicated by the arrows of the solid lines, the magnetic field has a plate shape wider than the image detector 1 on the imaging surface 5 and the rear surface of the imaging surface 5. As a result, an eddy current is generated by Lentz's law on the imaging surface 5 and the housing 2 on the rear surface of the imaging surface 5. This eddy current generates a magnetic field indicated by arrows of broken lines in a direction in which the irradiated magnetic field is canceled, and cancels the magnetic field of the Z component that is to come into the imaging surface 5. For this reason, the magnetic field intensity inside the housing 2 does not increase. That is, this structure makes it difficult for the magnetic field of the Z component perpendicularly coming into the imaging surface 5 to enter the inside of the housing 2, thereby suppressing the magnetic field component reaching the image detector 1 contained inside the housing 2.

FIG. 2B shows a case in which a magnetic field of the X component perpendicularly coming into the openings 3 and 3′ formed in the side surfaces of the housing 2 irradiates the housing 2, as indicated by arrows of solid lines. Upon irradiation with the magnetic field of the X component perpendicularly coming into the opening 3′, the magnetic field enters the inside of the housing from the opening 3′, passes through the internal space of the housing, as indicated by arrows of broken lines in FIG. 2B, and comes out of the housing from the opening 3 because the opening 3 is formed in a surface facing the opening 3′. In this manner, when the openings 3′ and 3 are formed in facing side surfaces of the housing 2, an external magnetic field enters the inside of the housing 2 through the openings 3′ and 3 serving as an entrance and exit, and passes through the inside of the housing. As a result, the external magnetic field reaches the image detector 1 inside the housing 2, and noise appears in a captured image.

FIG. 2C shows a case in which a magnetic field of the Y component entering the housing 2 from the near side on the drawing parallel to the longitudinal direction of the openings 3 and 3′ formed in the side surfaces of the housing 2 irradiates the housing 2, as indicated by arrows of solid lines. The magnetic field of the Y component is a vector component parallel to the longitudinal direction of the openings 3 and 3′ formed in the side surfaces. The magnetic field of the Y component enters the inside of the housing 2 from the near-side ends of the openings 3 and 3′ in the longitudinal direction on the drawing, as indicated by arrows of broken lines. The magnetic field component parallel to the opening enters the inside of the housing 2, as indicated by the arrows of the broken lines in FIG. 2C, under the influence of eddy currents concentrated around the openings 3 and 3′ of the housing 2 upon irradiation with the external magnetic field, a detailed description of which will be omitted. The magnetic field entering the inside of the housing 2 comes out of the housing 2 from the far sides of the openings 3 and 3′ on the drawing.

As described above, in the housing 2 having the openings 3 and 3′ formed in facing side surfaces, magnetic fields of the X and Y components serving as horizontal magnetic fields act as magnetic field components entering the inside of the housing 2 from the openings 3 and 3′. If the magnetic fields of the X and Y components reach the image detector 1 inside the housing 2, they cause a problem that horizontal-striped noise periodically appears in a captured image, as described in Description of the Related Art. To solve this problem, this embodiment has a feature in which the magnetic material 4 having an area wider than the projection area of the image detector 1 is arranged inside the housing 2, as shown in FIG. 1.

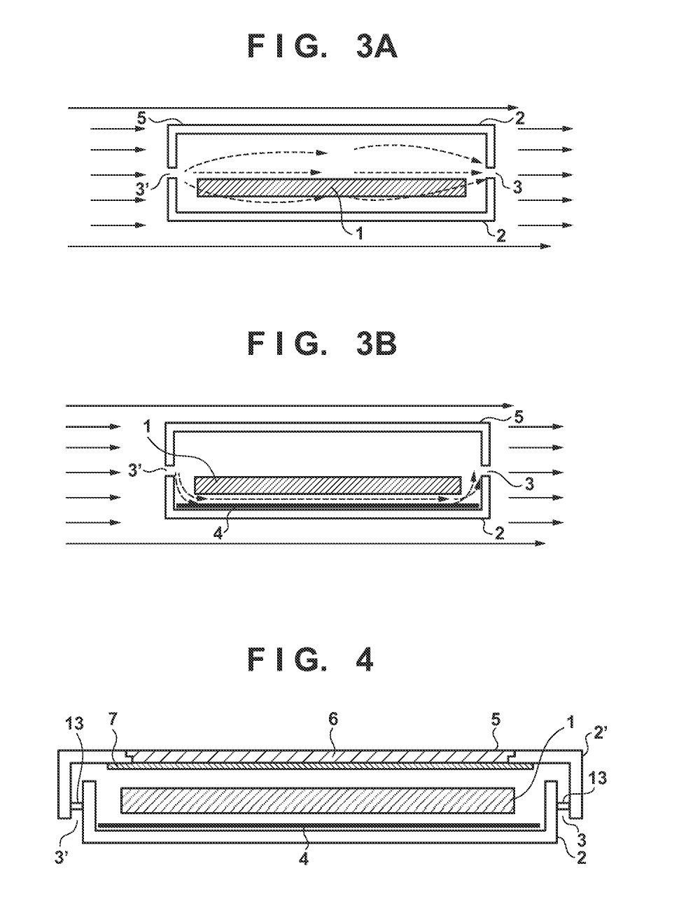

Next, an operation according to this embodiment will be described with reference to FIGS. 3A and 3B. FIGS. 3A and 3B are views for explaining the operation of the housing structure according to this embodiment. A magnetic field vector upon irradiation with a magnetic field of the X component is schematically indicated using arrows of broken lines. FIG. 3A shows a structure in which the magnetic material 4 is not arranged on the rear surface of the image detector 1 in the housing structure shown in FIG. 1. FIG. 3B shows a structure in which the magnetic material 4 having an area wider than the projection area of the image detector 1 is arranged on the rear surface of the image detector 1, as in the housing structure shown in FIG. 1.

In FIG. 3A, a magnetic field entering the housing 2 from the opening 3′ passes through the opening 3′, diffuses in the internal space of the housing 2, and passes through the inside of the housing 2. Then, the magnetic field concentrates at the opening 3, and comes out of the housing 2. At this time, if the magnetic field is superimposed on the image detector 1 contained in the housing 2, noise appears in a captured image.

Also in FIG. 3B, a magnetic field enters the housing 2 from the opening 3′, as in FIG. 3A. However, the magnetic material 4 having an area wider than the projection area of the image detector 1 is arranged on the rear surface of the image detector 1. Hence, an operation of attracting, to the magnetic material 4, the magnetic field entering the inside of the housing 2 in a space ranging from the opening 3′ to the end of the image detector 1 is generated, as indicated by arrows of broken lines in FIG. 3B. More specifically, the magnetic field entering the opening 3′ is attracted to the magnetic material 4 arranged on the rear surface of the image detector 1. The magnetic field attracted to the magnetic material 4 goes around the image detector 1 up to the side of the opening 3 facing the side surface of the opening 3′ along, as a magnetic path, the magnetic material 4 arranged on the rear surface of the image detector 1. The magnetic field going around the image detector 1 along the magnetic material 4 serving as the magnetic path travels away from the magnetic material 4 serving as the magnetic path in the space between the end of the image detector 1 and the opening 3, and comes out of the housing 2 from the opening 3.

In this way, according to this embodiment, the magnetic material 4 having an area wider than the projection area of the image detector 1 is arranged on the rear surface of the image detector 1. A magnetic field entering the inside of the housing from the opening 3′ is attracted by the magnetic material 4 in front of the image detector 1, and goes around the image detector 1. Thus, the magnetic field reaching the image detector 1 is reduced. Although a description will be omitted, even when a magnetic field of the Y component as described with reference to FIG. 2C irradiates the housing 2, the magnetic field reaching the image detector 1 is reduced because the magnetic material 4 has the operation of attracting a magnetic field entering the housing from the opening 3′, and the operation of causing the magnetic field go around the image detector 1, as described with reference to FIG. 2C.

Application examples of the first embodiment will be explained below. As the application examples, arrangements for further enhancing the effect of attracting, to the magnetic material 4, a magnetic field of the horizontal component entering the housing from the opening of the side surface, and making the magnetic field go around the image detector 1 will be explained. Note that imaging apparatuses shown in FIGS. 4 and 6 to 11 are stationary digital X-ray imaging apparatuses.

### Application Example 1-1

FIG. 4 shows the structure of an imaging apparatus according to application example 1-1. As will be described below, the effect of the imaging apparatus shown in FIG. 4 has actually been verified. In FIG. 4, the imaging apparatus includes a lower box housing 2 and upper box housing 2′ that contain the image detector 1. The lower box housing 2 and the upper box housing 2′ are made of a conductive material. The upper box housing 2′ has the imaging surface 5. A plastic material made of an X-ray transmission material is mated in the opening of the imaging surface 5 of the upper box housing 2′, which will be described below.

The magnetic material 4 wider than the projection area of the image detector 1 is arranged on the rear surface of the image detector 1. The openings 3 and 3′ are gaps at which the lower box housing 2 and the upper box housing 2′ overlap each other. Each of screws 13 mates (couples) two facing surfaces of the side surfaces on which the lower box housing 2 and the upper box housing 2′ overlap each other. In this state, conduction between the lower box housing 2 and the upper box housing 2′ is obtained. When the mating screws 13 are removed, the lower box housing 2 and the upper box housing 2′ can be easily disassembled. Gaps about 1 mm to 3 mm wide are formed on the four sides inside the lower box housing 2 and outside the upper box housing 2′ except for the portions mated by the screws 13. This implements a structure in which the inside of the housing ensures air permeability with the outside and heat is hardly confined inside.

CFRP (Carbon Fiber Reinforced Plastic) 6 excellent in X-ray transmittance is mated outside the opening of the imaging surface 5 of the upper box housing 2′. The inside of the opening is covered with an aluminum sheet 7 having a high X-ray transmittance and a small electrical resistance value, and conduction with the upper box housing 2′ is obtained on the four sides of the opening.

The reason why the CFRP 6 and the aluminum sheet 7 are used will be explained. At the time of imaging, a patient may directly contact the X-ray incident surface and add the weight. To prevent plastic deformation against the weight, the CFRP having characteristics excellent in strength and elasticity is suitable. Since the CFRP contains carbon, the electrical resistance value is small but is apparently larger than that of a metal, and no shield structure is formed. The aluminum sheet 7 having a high X-ray transmittance and a small electrical resistance value covers the opening from the inside of the housing, and conduction with the upper box housing is obtained on the four sides of the opening. As for the aluminum sheet 7 covering the opening of the imaging surface 5 from the inside of the upper box housing 2′, an aluminum sheet having a thickness of about 30 μm is generally used to suppress the X-ray attenuation factor.

As a supplemental explanation, as for the opening of the X-ray incident surface of the upper box housing 2′, magnetic fields of the horizontal components (the magnetic fields of the X and Y components) are cut off because conduction with the nonmagnetic metal housing (upper box housing 2′) is obtained on the four sides of the opening by the aluminum sheet covering the opening from the inside. When no aluminum sheet exists in this opening, if the magnetic fields of the horizontal components irradiate the housing, an eddy current generated in the nonmagnetic metal housing concentrates at the periphery of the opening, and the magnetic fields enter the inside of the housing owing to a magnetic field generated by the eddy current.

In this application example, the aluminum sheet is rendered conductive with the housing in the opening of the X-ray incident surface of the upper box housing 2′. Hence, entrance of the magnetic fields of the horizontal components from the opening of the upper box housing 2′ is prevented, and entrance of the horizontal magnetic fields is limited to entrance from the openings on the four sides of the overlapping side surfaces of the upper box housing 2′ and lower box housing 2. In the view of the structure of the imaging apparatus shown in FIG. 4, openings from which an external magnetic field enters the housing are the openings 3 and 3′ serving as the overlapping gaps between the lower box housing 2 and the upper box housing 2′. Since FIG. 4 is a sectional view, openings are only the openings 3 and 3′, but openings from which horizontal magnetic fields actually enter the housing are formed on all the four sides of the side surfaces.

To verify the effect of this application example, a 26-kHz sinusoidal current was applied to a 1 meter square loop coil available from TESEC, and magnetic fields of the horizontal components irradiated the imaging apparatus according to this application example. Then, amounts of image noise that appeared in captured images were compared. As the magnetic material 4, a high-permeability material FINEMET® available from Hitachi Metals was arranged. In practice, one 18-μm thick FINEMET sheet wider than the projection area of the image detector 1 was arranged as the magnetic material 4 on the rear surface of the image detector 1. As a result of comparing the image noise amounts, letting an image noise amount be 100% when no FINEMET sheet was arranged, an image noise amount obtained when the FINEMET sheet was arranged was reduced to 37%, and a 63% image noise reduction effect was confirmed.

Then, numerical analysis based on a three-dimensional electromagnetic field was performed to verify the reduction effect of external magnetic field noise reaching the inside of the housing based on the relative permeability of the magnetic material 4. Software used for analysis was Maxwell 3D commercially available from ANSYS. By using this software, the intensity of a magnetic field entering the inside of the housing was calculated. In analysis, as in actual measurement, the housing of the imaging apparatus shown in FIG. 4, and a 1 meter square loop coil that emitted external magnetic fields of the horizontal components were modeled, and the density of a magnetic flux reaching the inside of the housing was set to be a frequency of 26 kHz.

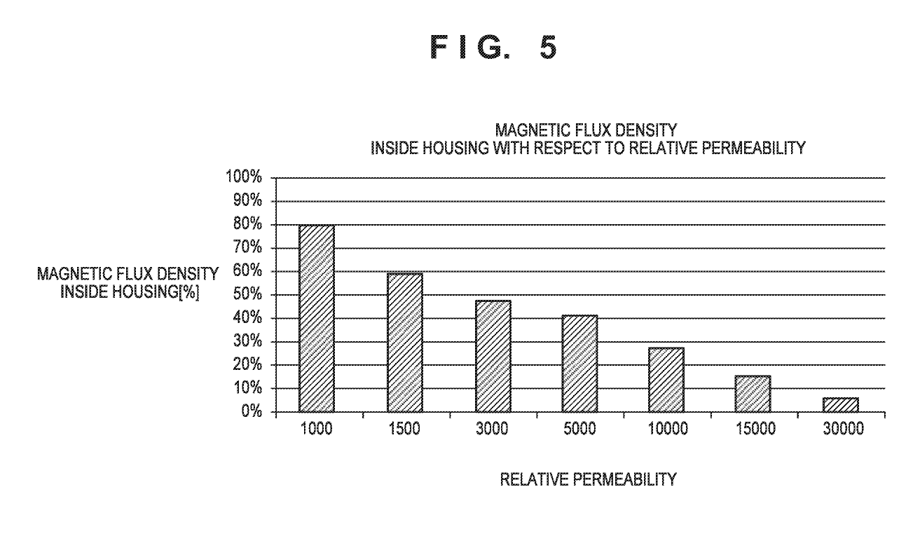

FIG. 5 shows the analysis result. FIG. 5 is a graph showing the magnetic flux density inside the housing with respect to the relative permeability serving as a parameter when the magnetic flux density inside the housing in the case in which no magnetic material is arranged inside the housing is defined as 100%. As is apparent from FIG. 5, as the relative permeability increases, a magnetic field reaching the inside of the housing is reduced. The result that the density of a magnetic flux reaching the inside of the housing became 50% or less at a relative permeability of 3,000 was obtained, compared to a case in which no magnetic material was arranged. Note that the analysis result in FIG. 5 is a result obtained when the density of a magnetic flux reaching the inside of the housing is a frequency of 26 kHz. However, even when the frequency of the magnetic flux density falls within the band of 1 kHz to 100 kHz, the same result is obtained. It is confirmed that the effect is obtained at least when the frequency of the density of a magnetic flux reaching the inside of the housing falls within the band of 1 kHz to 100 kHz and the relative permeability of the magnetic material 4 is 1,000 to 200,000.

### Application Example 1-2

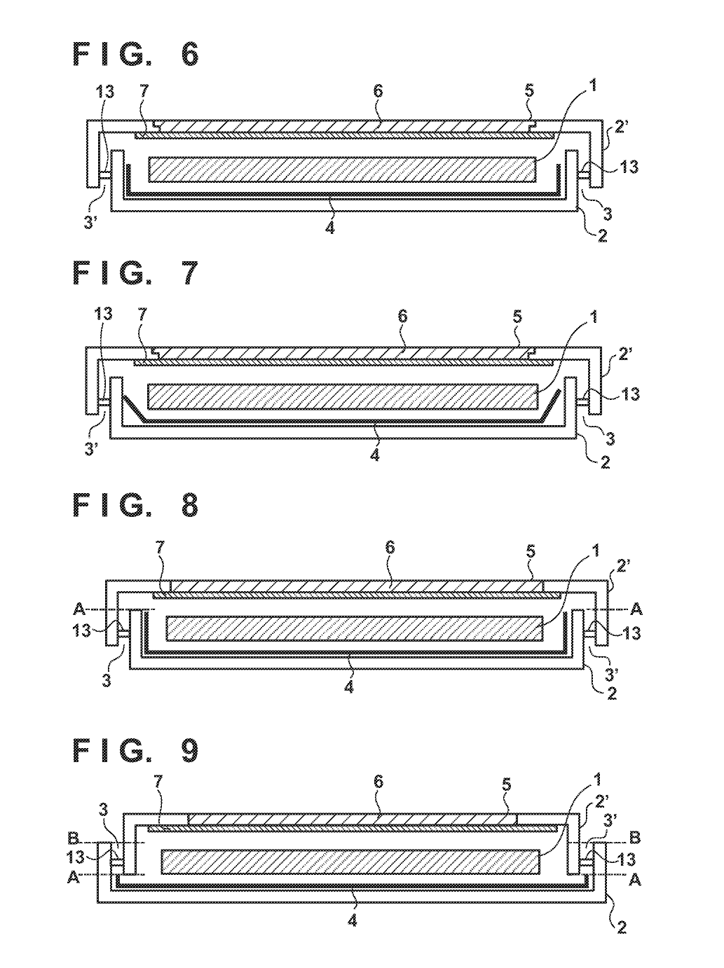

FIGS. 6 and 7 show the structure of an imaging apparatus according to application example 1-2. A difference from FIG. 4 for explaining application example 1-1 will be mainly explained. In FIGS. 6 and 7, the ends of the magnetic material 4 arranged on the rear surface of the image detector 1 stand toward the openings of the inner side surfaces of the lower box housing 2.

In FIG. 6, the magnetic material 4 arranged on the rear surface of the image detector 1 is vertically bent along the inner walls of the side surfaces of the lower box housing 2 with respect to side surfaces in which the openings of the lower box housing 2 are formed, and the ends of the magnetic material 4 stand toward the openings of the side surfaces. In FIG. 7, the magnetic material 4 arranged on the rear surface of the image detector 1 is bent at the ends of the image detector 1 with respect to side surfaces of the lower box housing 2 in which the openings are formed, and the ends of the magnetic material 4 stand toward the openings.

In this application example, to verify the image noise amount, a 18-μm thick FINEMET sheet, which was the same material as the material described in application example 1-1, was bent to stand toward the openings of the side surfaces, and was arranged as the magnetic material 4. In this arrangement, as in application example 1-1, magnetic fields of the horizontal components irradiated the imaging apparatus, and the image noise amounts of captured images were compared. As a result of comparing the image noise amounts, letting an image noise amount be 100% when no FINEMET sheet was arranged, both image noise amounts when the FINEMET sheet was arranged in the arrangements of FIGS. 6 and 7 were reduced to 30%, and a 70% image noise reduction effect was confirmed.

By arranging the magnetic material 4 with a structure in which it stands toward the openings of the inner side surfaces of the lower box housing 2, this improves the effect of attracting a magnetic field. As a result, a magnetic field reaching the image detector 1 decreased and the image noise amount of a captured image was also reduced. Note that the magnetic material 4 arranged toward the openings of the side surfaces of the lower box housing 2 may be divided. This is because the noise reduction amount did not differ between a case in which the magnetic material 4 on the rear surface of the image detector 1 was formed from one member and bent, and a case in which the magnetic material 4 on the side surface was divided from the magnetic material 4 on the rear surface, and the magnetic materials 4 were divisionally arranged for the rear surface and the side surface. In the divisional arrangement, it is desirable to arrange the divided magnetic materials close to each other so as not to increase the magnetic impedance because a magnetic path for go-around is formed.

### Application Example 1-3

FIGS. 8 and 9 show the structure of an imaging apparatus according to application example 1-3. A difference from FIG. 6 for explaining application example 1-2 will be mainly explained. In FIG. 6, the magnetic material 4 arranged on the rear surface of the image detector 1 is bent so that the ends of the magnetic material 4 stand toward the openings of the side surfaces of the lower box housing 2. In FIG. 8, the magnetic material 4 is arranged up to points A in the drawing serving as the opening inner ends of the side surfaces of the lower box housing 2.

In the verified digital X-ray imaging apparatus, the height of the inner wall of the side surface of the lower box housing 2 is 3 cm. The magnetic material 4 is vertically bent along the inner walls of the side surfaces of the lower box housing 2, and is arranged toward the openings of the side surfaces. In the arrangement shown in FIG. 8, as in application example 1-1 and application example 1-2, the image noise amount was examined by changing the height of the magnetic material 4. As a result of comparing image noise amounts, the reduction effect was confirmed to be −2% when the magnetic material 4 was bent by a height of 1 cm from the rear surface in this application example, −7% when it was bent by 2 cm, and −17% when it was bent by 3 cm, compared with a case in which the magnetic material 4 was only arranged on the rear surface in application example 1-1.

The above-described effect revealed that the magnetic material 4 bent toward the openings of the side surfaces was made to be higher than the flat portion of the magnetic material 4 arranged on the rear surface of the image detector 1, thereby enhancing the effect of further attracting a magnetic field entering the inside of the housing from the openings along the side surfaces of the housing. Note that the maximum effect is obtained when the magnetic material 4 is bent to a height indicated by point A serving as the opening inner ends of the side surfaces of the lower box housing 2, as shown in FIG. 8. However, it was confirmed that external noise reaching the image detector 1 is reduced as long as the height is the finishing accuracy of the magnetic material 4±1 mm with respect to the 3-cm height of the inner wall of the side surface of the lower box housing 2 in consideration of assembly.

In FIG. 9, the structure of the housing containing the image detector 1 is different from that in FIG. 8. More specifically, FIG. 9 shows a structure in which the lower box housing 2 containing the image detector 1 is larger in size than the upper box housing 2′, unlike the structure in FIG. 8. In the housing structure shown in FIG. 9, the magnetic material 4 arranged on the rear surface of the image detector 1 is arranged toward the openings of the side surfaces of the lower box housing 2, and is arranged in the overlapping openings 3 and 3′ of the lower box housing 2 and upper box housing 2′.

Numerical analysis based on a three-dimensional electromagnetic field was performed using, as a parameter, the height of the magnetic material 4 on the inner side surface of the lower box housing 2 in the housing structure shown in FIG. 9. Software used for analysis was Maxwell 3D, as in application example 1-1. By using this software, the intensities of magnetic fields entering the inside of the housing were calculated and compared. Note that the relative permeability of the arranged magnetic material 4 is set to be 15,000. In verification, three models, that is, the height of opening inner end A, the height of opening outer end B, and the intermediate height between inner end A and outer end B, as shown in FIG. 9, were created as heights of the magnetic material 4 on the side surface.

As a result of the verification, letting the magnetic field intensity inside the housing be 100% when the magnetic material 4 on the side surface was arranged up to the height of inner end A, the magnetic field intensity increased to 150% at the intermediate height between inner end A and outer end B, and increased to 225% at the height of outer end B. This is because, if the magnetic material 4 is arranged to be higher than the opening inner end and reach the inside of the opening, the magnetic material 4 attracts even an extra external magnetic field more than one entering the housing from the opening when the magnetic material 4 is not arranged, and the magnetic field intensity inside the housing is increased. It was therefore confirmed that when the magnetic material 4 on the side surface was arranged to the opening inner end, the effect of attracting a magnetic field entering the inside of the housing was maximized to reduce the magnetic field reaching the image detector 1.

### Application Example 1-4

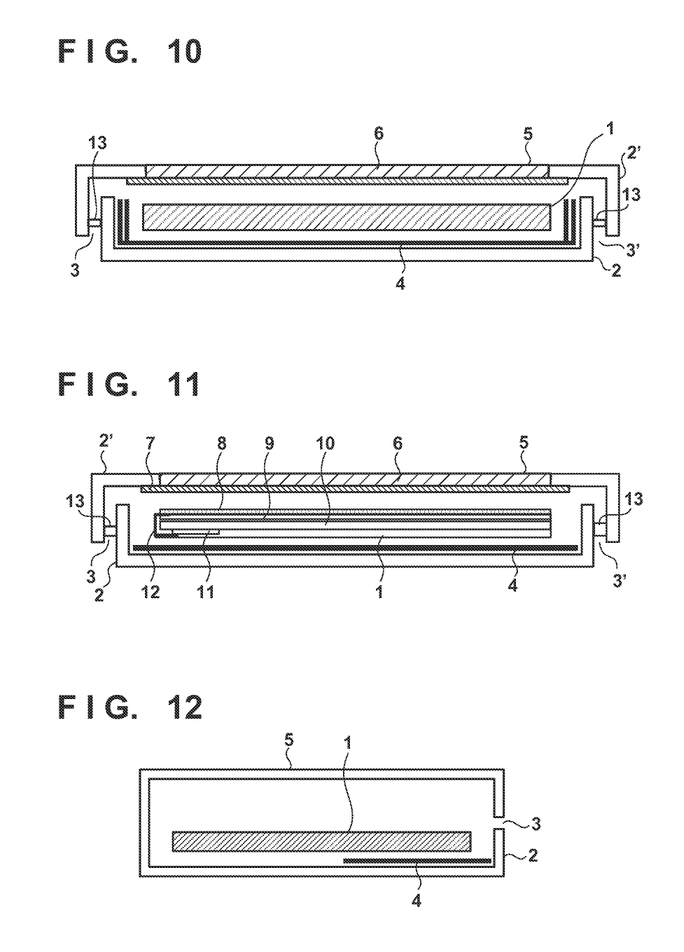

FIG. 10 shows the structure of an imaging apparatus according to application example 1-4. A difference from FIG. 4 for explaining application example 1-1 will be mainly explained. In FIG. 10, the number of magnetic materials 4 that were arranged on the rear surface of the image detector 1 in FIG. 4 and bent toward the openings of the side surfaces was increased, and the magnetic materials 4 were arranged to overlap each other. That is, the arrangement according to this application example is an arrangement in which the magnetic materials 4 are arranged toward the openings of the side surfaces of the lower box housing 2, and the number of magnetic materials 4 is increased.

In this arrangement, as in application example 1-1, magnetic fields of the horizontal components irradiated the imaging apparatus, and the image noise amounts of captured images were compared. Let the image noise amount be 100% when the magnetic material 4 was not arranged along the side surface, that is, when the magnetic material 4 existed on only the rear surface of the image detector 1. Then, reduction effects obtained when one, three, and five magnetic materials 4 were arranged on the side surface were verified. As a result of comparing the image noise amounts, the noise amount was reduced to 83% when one magnetic material 4 was arranged up to the opening inner end, 73% when three magnetic materials 4 were arranged, and 70% when five magnetic materials 4 were arranged. From this, when the magnetic materials 4 arranged toward the openings of the side surfaces of the housing are superimposed at least partially to increase the thickness, the effect of attracting a magnetic field entering the opening can be enhanced to reduce the magnetic field reaching the image detector 1.

The same effect is also obtained even when the number of magnetic materials 4 arranged on only the rear surface of the image detector 1 is increased without arranging the magnetic material 4 toward the opening of the side surface of the housing. Let an image noise amount be 100% when one 18-μm FINEMET sheet was arranged on the rear surface of the image detector 1 described in application example 1-1. Then, the image noise amount was reduced to 64% when two FINEMET sheets were arranged on the rear surface. From this, as the numbers of overlapping magnetic materials 4 arranged on the rear surface of the image detector 1 and overlapping magnetic materials 4 arranged toward the openings of the side surfaces of the housing are increased, the effect of attracting a magnetic field entering the housing from the opening can be enhanced to reduce the magnetic field reaching the image detector 1. The reduction effect is enhanced regardless of which of the number of magnetic materials 4 arranged on the rear surface and the number of magnetic materials 4 on the side surface is increased. By increasing the number of magnetic materials 4 on either side, the effect of reducing a magnetic field reaching the image detector 1 is enhanced. The same effect can be expected even when the thickness of the magnetic material 4 is increased. However, if the thickness is the same for a highly conductive magnetic material, the effect of attracting a magnetic field is enhanced by increasing the number of thin materials.

### Application Example 1-5

FIG. 11 shows the structure of an imaging apparatus according to application example 1-5. A difference from FIG. 4 for explaining application example 1-1 will be mainly explained. FIG. 11 is a view showing in detail the image detector 1 of FIG. 4. The image detector 1 is formed by stacking a scintillator 8 and a substrate 9 including photoelectric converters (not shown). As the substrate 9, a glass plate is often used because of necessities to not cause a chemical action with a semiconductor element, resist the temperature of a semiconductor process, and have dimensional stability and the like. The photoelectric converters are formed in a matrix on the substrate by a semiconductor process. The scintillator 8 is prepared by coating a resin plate with a phosphor of a metal compound, and is integrated and fixed to a base. The stacking order of the scintillator 8 and substrate 9 is arbitrary.

A circuit substrate 11 on which a signal processing unit and power supply circuit unit serving as driving circuit units constituted by electronic components configured to process a photoelectrically converted electrical signal are mounted is arranged on the rear surface of a support base 10. The circuit substrate 11 is connected to the substrate 9 by a flexible printed circuit board 12 and fixed to the support base 10. On the flexible printed circuit board 12, the semiconductor elements of a driver IC for read driving (not shown) of the photoelectric converters arrayed in a matrix, and an amplifier IC for amplifying a photoelectrically converted weak electrical signal are mounted as so-called TCP (Tape Carrier Package).

The image detector 1, especially, the substrate 9, circuit substrate 11, and flexible printed circuit board 12 handle a weak analog signal. Thus, when an external magnetic field is superimposed, noise appears in a captured image. The following arrangement is therefore employed to prevent a magnetic field entering the housing from the opening of the side surface of the conductive housing from reaching the image detector 1, especially, the substrate 9, circuit substrate 11, and flexible printed circuit board 12, and from being superimposed in an image signal.

A magnetic material having an area wider than the projection area of the image detector 1, a frequency of 1 kHz to 100 kHz, and a relative permeability of 1,000 to 200,000 is arranged on the rear surface of the image detector 1. With this arrangement, a magnetic field entering the housing from the opening of the side surface can be attracted to the magnetic material and go around the image detector 1. This produces an effect of reducing a magnetic field reaching the image detector and reducing even noise of a captured image.

### Second Embodiment

FIG. 12 shows the structure of an imaging apparatus according to the second embodiment. This structure is different from that shown in FIG. 1 described in the first embodiment in which the side surface of a conductive housing 2 has only an opening 3. A magnetic material 4 is arranged on the rear surface of an image detector 1 from the opening of the side surface of the housing up to half the image detector 1. Note that the magnetic material 4 is not limited to the arrangement as shown in FIG. 12, and may be arranged on the rear surface of the image detector 1 from the opening of the side surface of the housing up to almost half the image detector 1.

In a stationary X-ray imaging apparatus, an opening is formed in the side surface of a housing in order to insert/remove a scattered X-ray removal grid to/from the inside of the housing depending on an object or portion to be imaged. The first embodiment has described an example in which magnetic fields of the horizontal components enter the inside of the housing from openings on the four sides of side surfaces on which upper and lower box housings overlap each other, as described in application example 1-1 to application example 1-5. The second embodiment will explain the structure of a housing in which conduction is obtained by welding or the like in openings on the four sides of side surfaces on which upper and lower box housings overlap each other, so as to prevent entrance of magnetic fields of the horizontal components, then the openings are shielded, and an opening for inserting/removing the scattered X-ray removal grid is formed on one side of the side surface. Note that this arrangement assumes a product or the like highly resistant to moisture and dust.

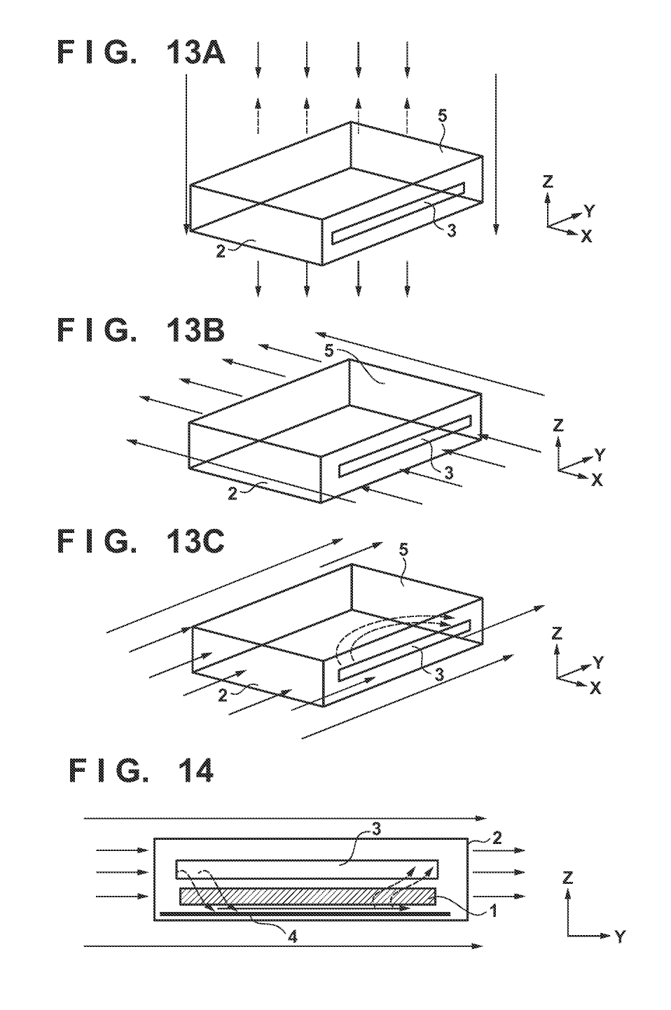

Even in the second embodiment, as in the first embodiment, an external magnetic field entering the inside of the housing when the magnetic field externally comes in will be explained with reference to FIGS. 13A to 13C. FIGS. 13A to 13C are views for explaining the influence of external magnetic field noise according to this embodiment.

FIG. 13A shows a case in which a magnetic field of the Z component perpendicularly coming into an imaging surface 5 irradiates the housing 2, as indicated by arrows of solid lines. In FIG. 13A, as in the description of FIG. 2A according to the first embodiment, when the magnetic field of the Z component comes into the housing 2, it is canceled by a demagnetizing field generated by an eddy current flowing through the imaging surface and its rear surface, so the magnetic field intensity inside the housing 2 does not increase.

FIG. 13B shows a case in which a magnetic field of the X component perpendicularly coming into the opening 3 formed in the side surfaces of the housing 2 enters the housing 2. In FIG. 13B, since the opening 3′ does not exist in a surface facing the opening 3′, the magnetic field of the X component is canceled by a demagnetizing field generated by an eddy current flowing through the side surface facing the opening 3, and the magnetic field intensity inside the housing 2 does not increase. When an opening is formed in the facing side surface of the housing 2 (examples of FIGS. 2A to 2C), the external magnetic field of the X component enters the inside of the housing 2 from the openings 3 and 3′ serving as an entrance and exit. However, when one opening is formed in the side surface, as shown in FIG. 13B, the X component perpendicularly coming into the opening 3 does not enter the inside of the housing 2.

FIG. 13C is a view for explaining a case in which a magnetic field of the Y component coming in parallel to the longitudinal direction of the opening 3 formed in the side surface of the housing 2 irradiates the housing 2. Similarly to the description of the first embodiment, the magnetic field of the Y component enters the inside of the housing 2 from the near side of the opening 3 on the drawing, as shown in FIG. 13C, and comes out of the housing 2 from the back side of the opening 3 on the drawing, as indicated by arrows of broken lines.

From this, when the side surface opening is formed on only one side of the side surface of the housing 2, as shown in FIG. 12, a magnetic field component entering the housing from the opening 3 is limited to a magnetic field of the Y component coming in parallel to the longitudinal direction of the opening 3. Further, when the side surface opening exists on only one side of the side surface, an opening from which a magnetic field enters the housing 2 and from which the magnetic field comes out of the housing 2 is the opening 3. Thus, the magnetic field intensity increases near the opening 3 and does not increase toward the back side from the opening 3. This is because no opening exists on a facing side surface, unlike the first embodiment, and there is no component passing through the inside of the housing from an opening to another opening, like the X component described in the first embodiment.

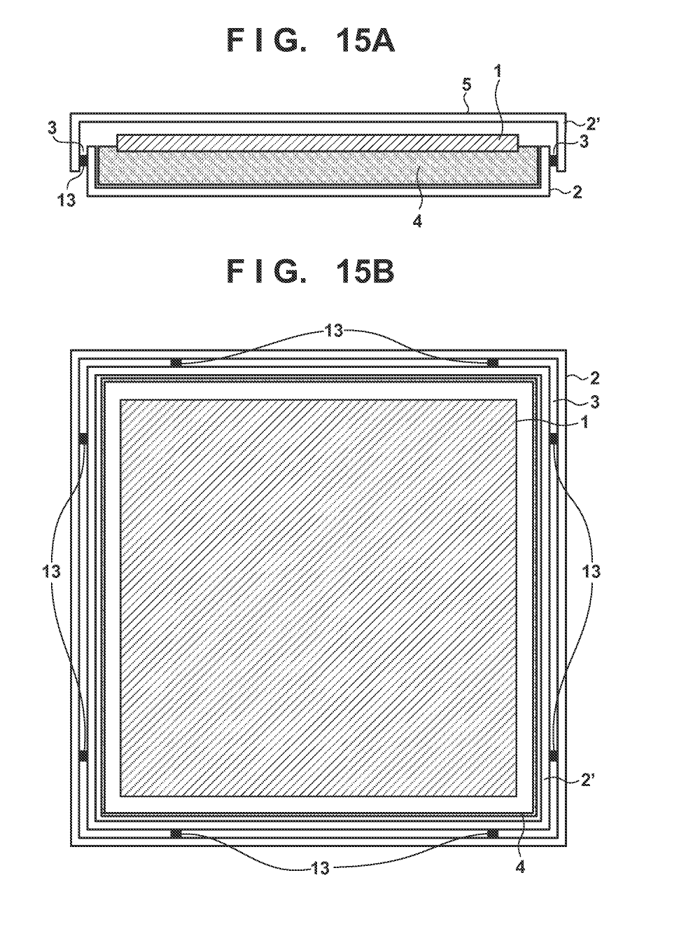

Next, an operation according to the second embodiment will be described with reference to FIG. 14. FIG. 14 is a view for explaining the operation of the housing structure. FIG. 14 is a sectional view from the side surface of the housing structure in which the opening 3 as shown in FIG. 12 is formed. For descriptive convenience, the image detector 1 and the magnetic material 4 are seen in FIG. 14. The magnetic material 4 is arranged from the opening 3 of the side surface up to a half surface having half the area of the image detector 1 on the rear surface of the image detector 1. Note that FIG. 14 is drawn on a Y-Z plane, as illustrated.

In FIG. 14, solid lines indicate the magnetic field vector of the Y component coming in parallel to the longitudinal direction of the opening 3, and arrows of broken lines indicate the vector of a magnetic field entering the housing from the opening. In FIG. 14, the magnetic field of the Y component coming in parallel to the longitudinal direction of the opening 3 enters the inside of the housing 2 from the left side of the opening 3 in the drawing, as indicated by arrows of broken lines, and comes out of the housing from the right side of the opening 3 in the drawing.

In FIG. 14, the magnetic field of the Y component enters the inside of the housing 2 from the left side of the opening 3 in the drawing. Since the magnetic material 4 is arranged up to the half surface of the image detector 1 from the side surface in which the opening 3 is formed, the magnetic field having passed through the opening 3 is attracted to the magnetic material 4 on a path from the opening 3 up to the image detector 1. The magnetic field attracted by the magnetic material 4 comes out of the housing 2 from the back side of the opening 3 in the drawing along the magnetic material 4 serving as a magnetic path on the rear surface of the image detector 1. Accordingly, the magnetic field reaching the image detector 1 is reduced. When the magnetic material 4 is not arranged, a magnetic field entering the housing from the opening 3 reaches even the image detector 1, and noise appears in a captured image.

The noise reduction effect by the housing structure of FIG. 14 was confirmed by numerical analysis based on a magnetic field intensity inside the housing when the magnetic material 4 was arranged from the opening of the side surface up to half the image detector 1 on the rear surface of the image detector 1. As software used for analysis, Maxwell 3D mentioned in application example 1-1 described above was used to calculate and compare magnetic field intensities inside the housing. In analysis, the magnetic material 4 was set to have a relative permeability of 15,000, a thickness of 18 μm, and dimensions of 516 mm×273 mm. The opening 3 of the side surface was an opening having a size 440 mm in the longitudinal direction and a height of 19 mm. As a result of the analysis, letting the magnetic field intensity inside the housing be 100% when the magnetic material 4 was not arranged from the opening of the side surface up to half the image detector 1 on the rear surface of the image detector 1, it was confirmed that the magnetic field intensity was reduced up to 50% when the magnetic material 4 was arranged.

As in the first embodiment, the second embodiment also implements the operation in which a magnetic field entering the inside of the housing from the opening of the side surface is attracted by the magnetic material 4 arranged on the rear surface of the image detector 1 and goes around before the external magnetic field reaches the image detector 1. The application examples described in the first embodiment are similarly applicable to the second embodiment. The effect of attracting, to the magnetic material, a magnetic field entering the housing from the opening can be further enhanced, and the magnetic field reaching the image detector can be reduced.

More specifically, even in the second embodiment, the magnetic material 4 is arranged toward the opening inside the housing 2 on the side surface having the opening 3. This improves the effect of attracting a magnetic field, decreases the magnetic field reaching the image detector 1 and thus reduces the noise amount of a captured image. By arranging, up to the opening inner end of the side surface, the magnetic material 4 that is arranged on the rear surface of the image detector 1 and bent toward the opening of the side surface, the effect of further attracting a magnetic field entering the housing 2 is enhanced. Further, the effect is obtained by increasing either the number of magnetic materials 4 on the rear surface of the image detector 1 or the number of magnetic materials 4 on the side surface. A magnetic field reaching the image detector can be reduced regardless of which of the number of magnetic materials 4 on the rear surface and the number of magnetic materials 4 on the side surface is increased.

### Third Embodiment

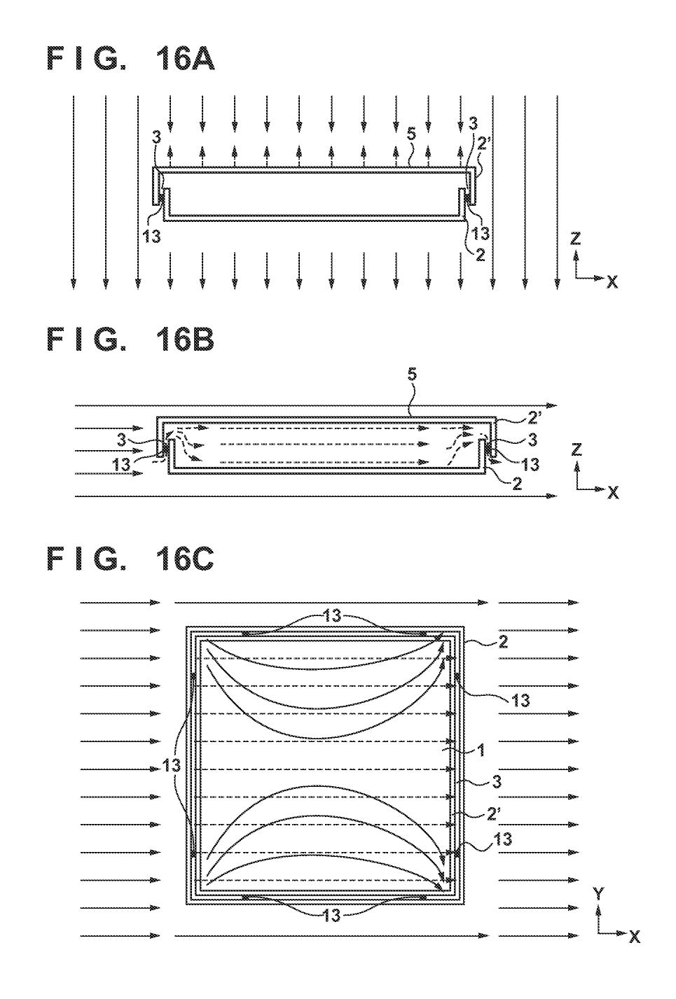

FIGS. 15A and 15B show the structure of an imaging apparatus according to the third embodiment. FIG. 15A is a sectional view of the imaging apparatus according to this embodiment when viewed from the side surface of the housing. FIG. 15B is a perspective view showing the imaging apparatus according to this embodiment when viewed from the imaging surface. The housing structure of the imaging apparatus according to this embodiment includes a conductive housing 2 that contains a planar image detector 1, and a conductive housing 2′ having an imaging surface 5.

The housing 2 has a lower box arrangement having a bottom surface and four sides of side surfaces, in order to contain the planar image detector. The housing 2′ has an upper box arrangement having the imaging surface 5 for receiving an X-ray, and four sides of side surfaces. The housing 2′ is configured to cover the housing 2. The housings 2 and 2′ have a structure in which they overlap each other on the four sides of the side surfaces. In this structure, openings are formed on the four sides of the side surfaces except for screws 13 that physically fix the housings 2 and 2′ and electrically obtain conduction. Magnetic materials 4 are arranged on the four sides of the side surfaces outside the periphery of the image detector 1 contained in a housing constituted by the housings 2 and 2′. The magnetic materials 4 are arranged along the inner side surfaces of the housing 2 so that the ends of the magnetic materials 4 are arranged from the opening ends of the inner side surfaces of the housing 2 toward the bottom surface of the housing 2.

Note that the housing 2 and the conductive housing 2′ are made of a conductive metal generally used in the exterior housing of a product, such as aluminum, stainless steel, or a steel sheet. The magnetic material 4 is made using a permalloy, amorphous alloy, FINEMET®, ferrite, or the like, which is a magnetic material having a relative permeability of 1,000 to 200,000 in a frequency band of 1 kHz to 100 kHz.

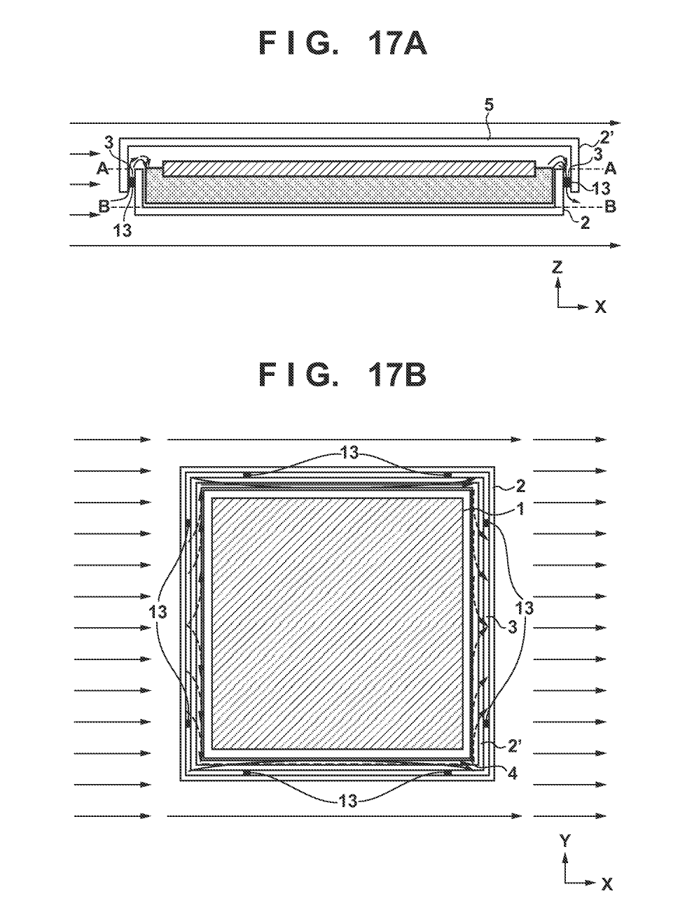

Next, magnetic fields externally entering the inside of the housing in the imaging apparatus having the housing structure described with reference to FIGS. 15A and 15B when the magnetic fields externally come in will be explained with reference to FIGS. 16A to 16C. FIGS. 16A to 16C are views for explaining the influence of external magnetic field noise according to this embodiment. In FIGS. 16A to 16C, the image detector 1 and magnetic material 4 contained inside the housing 2 in FIGS. 15A and 15B are omitted, in order to explain a magnetic field entering the inside of the housing when the external magnetic field comes in. As the influence of external magnetic field noise, the vector component of an external magnetic field coming into the imaging apparatus will be explained.

Magnetic fields from various directions enter the imaging apparatus in accordance with the installation position and the use state under the influence of radiation of a magnetic field from a device installed nearby or a large-power device, or leakage of a magnetic field. To clarify the explanation, an external magnetic field will be explained using spatial vectors along three, X-, Y-, and Z-axes. In this embodiment, a magnetic field of a vertical component perpendicularly coming into the imaging surface 5 is a Z component, and magnetic field components that are perpendicular to the Z component and perpendicularly come into the side surfaces of the housing are X and Y components. Since the left-and-right structure and top-and-bottom structure when viewed from the imaging surface are symmetrical structures in the housing according to this embodiment, the X and Y components of magnetic fields entering the inside of the housing are equal when they come in from the side surfaces of the housing. As a magnetic field component perpendicularly coming into the side surface of the housing, only the X component will be explained for convenience.

FIG. 16A is a view for explaining a case in which a magnetic field (arrows of solid lines) of the Z component perpendicularly coming in from the imaging surface 5 irradiates the housing 2. FIG. 16A is a sectional view when viewed from the side surface of the housing. Note that an incoming magnetic field is an AC component. In FIG. 16A, a magnetic field vector is expressed by arrows in one direction for convenience, and the kind of component of a magnetic field entering the inside of the housing will be explained. As shown in FIG. 16A, when a magnetic field of the Z component perpendicularly coming into the imaging surface 5 irradiates the housing, as indicated by the arrows of the solid lines, the magnetic field exists in a plate shape wider than the projection area of the image detector contained inside the housing on the imaging surface 5 and the bottom surface of the conductive housing 2 serving as the rear surface of the imaging surface. For this reason, when the magnetic field of the Z component perpendicularly coming into the imaging surface 5 irradiates the housing 2, an eddy current is generated by Lentz's law on the imaging surface 5 and the housing 2 serving as the bottom surface of the housing. This eddy current generates a magnetic field indicated by arrows of broken lines in a direction in which the irradiated magnetic field is canceled, and operates to cancel the magnetic field of the Z component that is to come into the imaging surface. As a result, the magnetic field intensity inside the housing does not increase. That is, the magnetic field of the Z component perpendicularly coming into the imaging surface 5 hardly enters the inside of the housing, thereby suppressing the magnetic field component reaching the image detector 1 contained inside the housing.

Next, a magnetic field component that is perpendicular to the Z component and perpendicularly comes into the side surface of the housing will be explained by the X component. FIGS. 16B and 16C are views for explaining a case in which a magnetic field (arrows of solid lines) of the X component perpendicularly coming into the side surfaces of the housing 2 and conductive housing 2′ irradiates the housing 2 and conductive housing 2′. FIG. 16B is a sectional view when viewed from the side surface of the housing. FIG. 16B shows a case in which a magnetic field perpendicularly irradiates the housing from the left side surface in the drawing, as indicated by the arrows of the solid lines. When the magnetic field of the X component perpendicularly coming into the side surface of the housing irradiates the side surface having the left opening 3 in the drawing, the magnetic field enters the inside of the housing from the left opening 3, as indicated by the arrows of the solid lines. Similarly, the opening 3 is formed in the right side surface that is a surface facing the irradiated side surface. Thus, the magnetic field entering the housing from the left opening 3 of the side surface passes through the opening, as indicated by arrows of broken lines, diffuses in the housing, and passes through the inside of the housing from left to right in the drawing. Then, the magnetic field concentrates at the right opening 3, and comes out of the housing.

FIG. 16C is a perspective view showing the imaging apparatus when viewed from the imaging surface. FIG. 16C shows a case in which a magnetic field perpendicularly irradiates the housing from the left side surface of the housing in the drawing, as in FIG. 16B. When the magnetic field irradiates the side surface having the left opening 3 in FIG. 16C, the magnetic field enters the inside of the housing from the gap of the left opening 3. Similarly, the opening 3 is formed in the right side surface serving as a facing surface. As described with reference to FIG. 16B, the magnetic field passes through the internal space of the housing, as indicated by arrows of broken lines, and comes out of the housing from the right opening 3. Also, as indicated by arrows of solid lines in FIG. 16C, the magnetic field enters the inside of the housing through the openings 3 of the upper and lower side surfaces in FIG. 16C from the left side in the drawing that is irradiated with the magnetic field.

Although a detailed description will be omitted, an external magnetic field enters the inside of the housing as indicated by the arrows of the solid lines in the drawing from the upper and lower openings 3 in the drawing under the influence of an eddy current concentrated at the periphery of the opening of the housing upon irradiation with the external magnetic field. The magnetic field entering the inside of the housing from the upper and lower openings 3 comes out of the housing from the right opening 3 in FIG. 16C. An actual magnetic field intensity inside the housing has an intensity distribution obtained by combining a magnetic field, indicated by the arrows of the broken lines, which enters the housing from the left opening 3 in FIG. 16C and comes out to the right opening 3, and a magnetic field, indicated by the arrows of the solid lines, which enters the housing from the left sides of the upper and lower openings 3 in the drawing and comes out to the right side of the upper and lower openings. In this fashion, when the openings are formed on the four sides of the side surfaces of the housing, external magnetic fields of the horizontal components enter the inside of the housing from the openings 3 on all the four sides and pass through the inside of the housing. Hence, the external magnetic field reaches the image detector 1 inside the housing, and noise appears in a captured image.

As described above, in the conductive housing having openings formed on the four sides of the side surfaces, magnetic fields of the horizontal components serve as magnetic field components entering the inside of the housing from the openings 3. If the magnetic fields of the horizontal components reach the image detector 1 inside the housing, horizontal-striped noise periodically appears in a captured image, as described in Description of the Related Art.

Next, an operation according to this embodiment will be described with reference to FIGS. 17A and 17B. FIGS. 17A and 17B are views corresponding to FIGS. 16A and 16B, respectively. The magnetic materials 4 are arranged on the four sides of the side surfaces outside the periphery of the image detector 1. The magnetic materials arranged on the four sides of the side surfaces are arranged from the opening ends of the inner side surfaces of the housing up to the bottom surface of the housing on the inner side surfaces of the housing, as shown in the sectional view of FIG. 17A when viewed from the side surface of the housing.

As shown in the sectional view of FIG. 17A when viewed from the side surface of the housing, a magnetic field entering the housing from the opening 3 of the left side surface in the drawing tries to diffuse inside the housing after passing through the opening 3. However, the magnetic material 4 arranged from the opening end of the inner side surface of the housing generates an operation of attracting the magnetic field to the magnetic material 4, as indicated by arrows of solid lines. As shown in the perspective view of FIG. 17B when viewed from the imaging surface, the magnetic field that has entered the housing from the left opening 3 in the drawing and has been attracted to the magnetic material 4 travels along the magnetic material 4 serving as a magnetic path upward or downward in the drawing, as indicated by arrows of broken lines. Further, the magnetic field attracted up to the right magnetic material 4 in the drawing along the upper or lower magnetic material 4 in FIG. 17B serving as a magnetic path goes around the magnetic material 4 on the right side surface in FIG. 17B. Magnetic fields entering the housing from the upper and lower openings 3 in FIG. 17B are attracted to the magnetic materials 4, and pass by the right magnetic material 4 in the drawing along the magnetic materials 4 serving as magnetic paths. A magnetic field entering the housing from the opening 3 of the side surface of the housing is attracted to the magnetic material 4 at the opening end of the inner side surface of the housing, travels along the magnetic material 4 serving as a magnetic path, then travels apart from the right magnetic material 4 in FIGS. 17A and 17B, and comes out of the housing from the right opening 3 in the drawings.

As described above, the magnetic materials 4 are arranged outside the periphery of the image detector 1 inside the conductive housing having openings formed on the four sides of the side surfaces. The ends of the magnetic materials 4 are arranged at the opening ends of the side surfaces of the housing inside the housing. A magnetic field entering the housing from the opening of the side surface is attracted before it reaches the image detector 1. Further, the magnetic field passes by the magnetic material 4 until the entering magnetic field comes out of the housing along the magnetic material 4 serving as a magnetic path. Thus, the magnetic field reaching the image detector 1 is reduced.

### Application Example 3-1

FIG. 18 is a view for explaining application example 3-1. FIG. 18 is a sectional view schematically showing a current stationary digital X-ray imaging apparatus, the effect of which has been actually verified, when viewed from the side surface. A housing 2 is a lower box housing that contains the image detector 1. A housing 2′ is an upper box housing that has the imaging surface 5 for receiving an X-ray and is configured to cover the lower box housing 2. The lower box housing 2 and the upper box housing 2′ are made of a steel sheet of a conductive material. The lower box housing 2 and the upper box housing 2′ overlap each other on the four sides of the side surfaces. Facing surfaces on the four sides of the overlapping side surfaces are mated by screws 13 to obtain conduction between the upper and lower housings. This implements a structure in which openings are formed on the four sides of the side surfaces except for the screws 13 that physically fix the lower box housing 2 and the upper box housing 2′ and electrically obtain conduction. This housing structure makes it easy to disassemble the lower box housing 2 and the upper box housing 2′ by removing the mating screws 13. Gaps about 1 mm to 3 mm wide are formed on the four sides of the side surfaces of the lower and upper boxes except for the portions mated by the screws. This implements a structure in which the inside of the housing ensures air permeability with the outside and heat is hardly confined inside.

CFRP (Carbon Fiber Reinforced Plastic) 6 excellent in X-ray transmittance is mated in the opening of the imaging surface 5 outside the housing. The opening is covered from the inside of the housing with an aluminum sheet 7 having a high X-ray transmittance and a small electrical resistance value, and conduction with the upper box housing is obtained on the four sides of the opening. At the time of imaging, a patient may directly contact the X-ray incident portion and add the weight. To prevent plastic deformation against the weight, the CFRP having characteristics excellent in strength and elasticity is suitable. Since the CFRP contains carbon, the electrical resistance value is small but is apparently larger than that of a metal, and no shield structure is formed. The aluminum sheet 7 having a high X-ray transmittance and a small electrical resistance value covers the opening from the inside of the housing, and conduction with the upper box housing is obtained on the four sides of the opening. As for the aluminum sheet 7 covering the opening of the imaging surface from the inside of the housing, an aluminum sheet having a thickness of about 30 μm is generally used to suppress the X-ray attenuation factor.

As a supplemental explanation, as for the opening of the X-ray incident surface of the upper box, magnetic fields of the horizontal components are cut off because the aluminum sheet covering the opening obtains conduction with the nonmagnetic metal housing on the four sides of the opening. When there is neither the housing nor the aluminum sheet in this opening, if the magnetic fields of the horizontal components irradiate the housing, an eddy current generated in the nonmagnetic metal housing concentrates at the periphery of the opening, and the magnetic fields enter the inside of the housing owing to a magnetic field generated by the eddy current. In this embodiment, the 30-μm aluminum sheet is rendered conductive with the housing in the opening of the X-ray incident surface of the upper box. Therefore, entrance of the magnetic fields of the horizontal components from the opening of the upper box is greatly reduced, and is limited to entrance of the horizontal magnetic fields from the openings on the four sides of the overlapping side surfaces of the upper and lower boxes.

As for an external magnetic field, a 26-kHz sinusoidal current was applied to a 1 meter square loop coil available from TESEC, and magnetic fields of the horizontal components irradiated the imaging apparatus. Then, amounts of noise that appeared in captured images were compared. As the magnetic material 4, a high-permeability material FINEMET® available from Hitachi Metals was arranged to verify the effect. In practice, FINEMET sheets each having a side surface height of 32.5 mm and a thickness of 18 μm were arranged by 468 mm one by one on the four sides of the side surfaces from inner side surface opening end A (FIG. 17A) of the side surface of the lower box housing 2 outside the periphery of the image detector 1 up to the bottom surface (bottom surface B in FIG. 17A) of the conductive housing 2. As a result of the verification, letting an image noise amount be 100% when no FINEMET sheet was arranged, an image noise amount obtained when the FINEMET sheet was arranged was reduced to 65%, and a 35% image noise reduction effect was confirmed.

As for the relative permeability, height, length, and thickness of the magnetic material 4, the reduction effect of external magnetic field noise reaching the inside of the housing was verified by numerical analysis based on a three-dimensional electromagnetic field. Software used for analysis was Maxwell 3D commercially available from ANSYS, and the intensity of a magnetic field entering the inside of the housing was calculated. As in actual measurement, the housing of a stationary digital X-ray imaging apparatus, and a 1 meter square loop coil that emitted external magnetic fields of the horizontal components were modeled, and the density of a magnetic flux reaching the inside of the housing was calculated at a frequency of 26 kHz. As the magnetic materials 4, magnetic materials each having a side surface height of 32.5 mm and a thickness of 18 μm were arranged by 468 mm on the four sides of the side surfaces. The intensity of a magnetic field entering the inside of the housing was calculated using the relative permeability as a parameter, and the magnetic field reaching the image detector 1 was confirmed.

FIG. 19 shows the calculation result. FIG. 19 is a graph showing the magnetic flux density inside the housing using the relative permeability as a parameter when the magnetic flux density inside the housing in the case in which the magnetic material 4 is not arranged inside the housing is defined as 100%. It was confirmed that a magnetic field reaching the inside of the housing was reduced as the relative permeability increased. The result that the density of a magnetic flux reaching the inside of the housing became 85% or less at a relative permeability of 1,000, compared to a case in which the magnetic material 4 was not arranged, was obtained.

Then, numerical analysis based on a three-dimensional electromagnetic field was performed to verify the reduction effect of external magnetic field noise reaching the image detector 1 when the magnetic materials 4 were arranged from the opening ends of the housing and when the magnetic materials 4 were arranged from the bottom surface of the housing in cases in which the height (Z direction) of the magnetic materials arranged on the four sides of the side surfaces was 10 mm and 20 mm. The length (X or Y direction) of the magnetic material 4 was 468 mm on each side surface. The reduction effect was confirmed by setting external magnetic field noise to be 100% when the magnetic materials 4 were not arranged on the four sides of the side surfaces outside the periphery of the image detector 1, that is, when no magnetic material 4 was arranged. In the case in which the height of the magnetic material 4 was 10 mm, noise was reduced to 77% when the magnetic materials 4 were arranged from the opening ends, but was reduced to only 90% when the 10-mm magnetic materials 4 were arranged from the bottom surface of the housing. In the case in which the height of the magnetic material 4 was 20 mm, noise was reduced to 64% when the magnetic materials 4 were arranged from the opening ends, but was reduced to only 73% when the magnetic materials 4 were arranged from the bottom surface of the housing.

These verification results revealed that the magnetic materials 4 arranged on the four sides of the side surfaces outside the periphery of the image detector had a high effect of attracting, to the magnetic materials 4, a magnetic field entering the housing from the openings 3 when the magnetic materials 4 were arranged from the opening ends of the inner side surfaces of the housing. Also, it was confirmed that a magnetic field reaching the image detector was reduced much more as the magnetic material 4 was higher.

Then, numerical analysis based on a three-dimensional electromagnetic field was performed to verify the reduction effect of external magnetic field noise reaching the image detector inside the housing in accordance with the length of the magnetic material 4. The height of the magnetic material 4 was 32.5 mm, the thickness was 18 μm, and the length was adopted as a parameter. The verification was performed by changing the length of the magnetic materials arranged on the four sides of the side surfaces to be 100 mm, 300 mm, and 400 mm centered on the center of each side. As for a magnetic field reaching the image detector 1, a partially high magnetic field reached the image detector 1, and no effect was confirmed. However, by setting the length of the magnetic material 4 to be equal to or larger than the length of the side surface of the image detector, the reduction effect was confirmed for the distribution of a magnetic field reaching the image detector.

These verification results revealed that no effect was exerted when the length of the magnetic materials 4 arranged on the four sides of the side surfaces outside the periphery of the image detector was equal to or smaller than the length of the side surface of the image detector.

Next, numerical analysis based on a three-dimensional electromagnetic field was performed to confirm the effect of the thickness (overlapping) of the magnetic materials 4 arranged on the side surface. The reduction effect in cases in which the number of magnetic materials 4 on the side surface was one and two was verified by setting noise to be 100% when the magnetic materials 4 were not arranged on the four sides of the side surfaces outside the periphery of the image detector, that is, when no magnetic material 4 was arranged. The length of the magnetic material 4 was 468 mm on each side surface, the thickness was 18 μm, and the number of magnetic materials was adopted as a parameter. The noise amount was reduced to 54% when one magnetic material 4 was arranged up to the opening inner end, and 36% when two magnetic materials 4 were arranged.

These verification results revealed that, as the magnetic materials 4 arranged on the four sides of the side surfaces outside the periphery of the image detector 1 were superimposed to increase the thickness, the effect of attracting a magnetic field entering the housing from the opening was enhanced, and the magnetic field reaching the image detector 1 could be reduced.

### Application Example 3-2

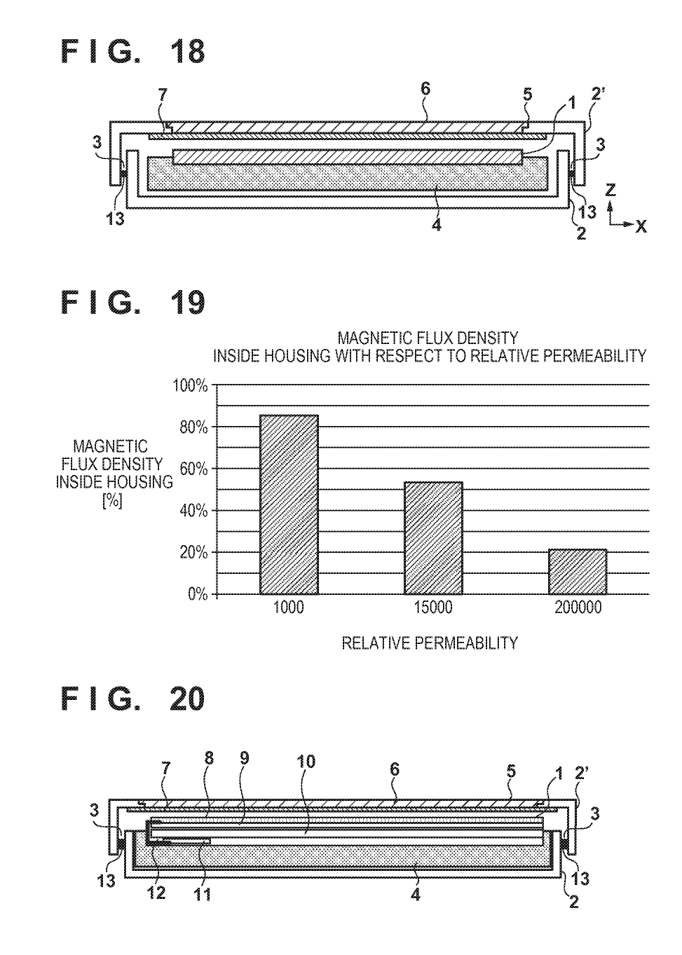

FIG. 20 is a graph for explaining application example 3-2. A difference from FIG. 19 explained in application example 3-1 will be mainly explained. The image detector 1 is formed by stacking a scintillator 8 and a substrate 9 including photoelectric converters (not shown). As the substrate 9, a glass plate is often used because of necessities to not cause a chemical action with a semiconductor element, resist the temperature of a semiconductor process, and have dimensional stability and the like. The photoelectric converters are formed in a matrix on the substrate 9 by a semiconductor process. The scintillator 8 is prepared by coating a resin plate with, for example, a phosphor of a metal compound, and is integrated and fixed to a base. The stacking order of the scintillator 8 and substrate 9 is arbitrary.

A circuit substrate 11 on which a signal processing unit and power supply circuit unit serving as driving circuit units constituted by electronic components configured to process a photoelectrically converted electrical signal are mounted is arranged on the rear surface of a support base 10. The circuit substrate 11 is connected to the substrate 9 by a flexible printed circuit board 12 and fixed to the support base 10. On the flexible printed circuit board 12, the semiconductor elements of a driver IC for read driving (not shown) of the photoelectric converters arrayed in a matrix, and an amplifier IC for amplifying a photoelectrically converted weak electrical signal are mounted as so-called TCP (Tape Carrier Package).

The image detector 1, especially, the substrate 9, circuit substrate 11, and flexible printed circuit board 12 handle a weak analog signal. Thus, when an external magnetic field is superimposed, noise appears in a captured image. The following arrangement is therefore employed to prevent a magnetic field entering the housing from the opening of the side surface of the housing from reaching the image detector 1, especially, the substrate 9, circuit substrate 11, and flexible printed circuit board 12, and from being superimposed in an image signal. More specifically, magnetic materials are arranged on the four sides of the side surfaces outside the periphery of the image detector 1. Here, the end of the magnetic material 4 on the inner side of the side surface of the housing is arranged from the opening end of the inner side surface of the housing toward the bottom surface of the housing.

With this arrangement, a magnetic field entering the opening of the side surface is attracted to the magnetic material 4, and passes by the magnetic material arranged outside the periphery of the image detector 1. This produces an effect of reducing a magnetic field reaching the image detector 1 and reducing even noise of a captured image. The arrangement of the housing according to this embodiment may be an arrangement in which the housing is not separated into upper and lower boxes, as shown in FIG. 1 or 12.

As described above, the third embodiment can implement a structure almost free from the influence of external noise by arranging a magnetic material on the rear surface of an imaging apparatus and appropriately setting the size and arrangement position of the magnetic material even in a structure in which a gap or opening is formed in the housing of the imaging apparatus. Note that the imaging apparatus according to each of the above-described embodiments has been explained as a digital X-ray imaging apparatus, but may be a digital radiation imaging apparatus using another radiation. Also, the magnetic material may have a shape other than the planar shape.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-109428, filed May 27, 2014 which is hereby incorporated by reference herein in its entirety.

What is claimed is:

1. An imaging apparatus comprising a housing including, on a side surface, at least one portion lower in magnetism shielding performance than a remaining portion of said housing, and configured to contain an image detector,
wherein the imaging apparatus includes a magnetic material, and
the magnetic material is arranged at a position between the image detector and the side surface including the portion, lower in magnetism shielding performance, of said housing, and a side of a rear surface of the image detector.

2. The apparatus according to claim 1, wherein the portion lower in magnetism shielding performance includes an electrical or physical opening.

3. The apparatus according to claim 2, wherein an end of the magnetic material is arranged to extend toward the opening of said housing.

4. The apparatus according to claim 3, wherein the end of the magnetic material is arranged to reach a position not higher than an opening end of said housing.

5. The apparatus according to claim 1, wherein the magnetic material is arranged with a portion along the side surface of said housing.

6. The apparatus according to claim 1, wherein the magnetic material is bent between the image detector and said housing and arranged.

7. The apparatus according to claim 1, wherein the magnetic material is divided and arranged between the image detector and said housing.

8. The apparatus according to claim 1, wherein the magnetic material includes a first magnetic material arranged between the image detector and the side surface including the portion, lower in magnetism shielding performance, of said housing, and a second magnetic material arranged on a side of a rear surface of the image detector.

9. The apparatus according to claim 1, wherein the magnetic material is planar.

10. The apparatus according to claim 9, wherein a surface of the magnetic material arranged on the rear surface of the image detector has an area wider than an area by which the rear surface of the image detector is occupied.

11. The apparatus according to claim 1, wherein the magnetic material includes magnetic materials that are superimposed and arranged at at least one portion of the magnetic material.

12. The apparatus according to claim 1, wherein the magnetic material has a relative permeability of 1,000 to 200,000.

13. The apparatus according to claim 1, wherein the image detector includes an X-ray detector.

14. An imaging apparatus comprising a housing configured to contain an image detector,
wherein said housing is constituted by an upper box housing and a lower box housing, and
a magnetic material arranged on a side of a rear surface of the image detector that faces the lower box housing in a state in which the upper box housing and the lower box housing are coupled has an end arranged at a position between the image detector and the side surface of the lower box housing.

15. The apparatus according to claim 14, wherein
the upper box housing is smaller in size than the lower box housing, and
the end of the magnetic material is arranged to extend toward an end of a side surface of the lower box housing.

16. The apparatus according to claim 15, wherein the end of the magnetic material is arranged to reach a position not higher than the end of the side surface of the upper box housing.

17. The apparatus according to claim 14, wherein
the upper box housing is larger in size than the lower box housing, and
the end of the magnetic material is arranged to extend toward the end of the side surface of the lower box housing.

18. The apparatus according to claim 17, wherein the end of the magnetic material is arranged to reach a position not higher than the end of the side surface of the lower box housing.

19. The apparatus according to claim 14, wherein the magnetic material is arranged including a side surface of a portion at which the upper box housing and the lower box housing are coupled.

20. An imaging apparatus comprising a housing configured to contain an image detector,
wherein said housing is constituted by an upper box housing and a lower box housing, and
a magnetic material is arranged along a side surface of the image detector between an internal side surface of the lower box housing and the image detector in a state in which the upper box housing and the lower box housing are coupled.
